(19) United States
(12) Patent Application Publication
(10) Pub. No.: US 2026/0363529 A1
(43) Pub. Date: Nov. 19, 2026

(54) ENDOSCOPE AND INSERTION TUBE ASSEMBLY THEREFOR

(71) Applicant: MACROLUX MEDICAL TECHNOLOGY CO., LTD., Shenzhen, Guangdong (CN)

(72) Inventors: Hanzhi Pan, Shenzhen, Guangdong (CN); Jin Wang, Shenzhen, Guangdong (CN)

(21) Appl. No.: 19/153,967

(22) PCT Filed: Oct. 26, 2023
(86) PCT No.: PCT/CN2023/126921

§ 371 Date: Aug. 6, 2025

(51) Int. Cl. A61B 1/00; A61B 1/018; A61B 1/05

(52) U.S. Cl. CPC A61B 1/00101; A61B 1/00105; A61B 1/018; A61B 1/053; A61B 1/00066; A61B 1/0011

(57) ABSTRACT

Disclosed are an endoscope and an insertion tube assembly thereof. The insertion tube assembly comprises: a main-body segment, having a first channel therein; an open-port segment, having therein a second channel and a third channel therein that are isolated from each other, with at least a portion of the second channel being arranged side-by-side with the third channel along the radial direction of the open-port segment, and an instrument outlet in communication with the second channel being provided on the outer peripheral surface of the open-port segment, the third channel being communicated with the first channel; a tip segment, having a fourth channel therein, with a through-flow port provided on the distal end face of the tip segment, and the proximal end of the fourth channel communicated with the third channel; an imaging module; and an instrument channel tube disposed inside the first channel.

### TECHNICAL FIELD

The present disclosure generally relates to an endoscope and an insertion tube assembly thereof.

### BACKGROUND OF THE DISCLOSURE

With the rapid advancement of medical technologies, endoscopes minimally invasive or non-invasive examinations and/or treatments have gained widespread adoption. An endoscope typically comprises an insertion tube assembly, a handle assembly, and an umbilical cable. The outer diameter of the insertion tube assembly is generally minimized to reduce patient discomfort and improve procedural compliance when advancing the tube through natural orifices or minimally invasive surgical pathways to target sites. Upon reaching the target site, tissue observation is enabled. When abnormal tissues are detected, instruments such as biopsy forceps or surgical scissors may be introduced through the instrument channel on the endoscope to perform biopsy or therapeutic devices may be deployed to treat lesions. A larger inner diameter of the instrument channel permits passage of instruments with greater outer diameters and diversity, thereby expanding biopsy and therapeutic capabilities.

Furthermore, when advancing an endoscope through a natural orifice, the lumen is typically in a collapsed state. This necessitates a fluid inflow/outflow assembly integrated into the endoscope. By infusing fluid through dedicated lumens, the collapsed lumen can be distended to establish a working space, thereby preventing the distal tip from abrading the mucosal lining or inflicting mechanical damage to the lumen. Moreover, distention of internal cavities is prerequisite for the endoscopic imaging system to achieve unobstructed visualization of pathological tissues. Consequently, an irrigation (inflow) channel requires a sufficient cross-sectional area to achieve requisite flow rates for effective cavity expansion. Given that human tissues exhibit limited tolerance to distention pressure, a concurrent drainage (outflow) channel must be integrated to maintain safe pressure levels. This is particularly crucial in uterine examination and therapy. The endoscope must traverse the constricted cervical os, followed by rapid uterine distension upon intrauterine access. Clear visualization is attainable only post-distention. Subsequently, the clinician advances/retracts, deflects, or rotates the scope body to survey the uterine cavity comprehensively. Upon detecting abnormal tissues, biopsy forceps, scissors, or other therapeutic instruments are deployed via the instrument channel for intervention. Therefore, in such special uterine applications, the endoscope requires: (1) a reduced outer diameter of the insertion tube, an enlarged inner diameter of the instrument channel, and both an irrigation (inflow) channel and a drainage (outflow) channel to enable rapid uterine distension.

In summary, contemporary endoscopes must concurrently satisfy: (i) a reduced outer diameter of the insertion tube assembly, (ii) an enlarged inner diameter of the instrument channel, (iii) a sufficiently sized irrigation (inflow) channel, and (iv) a sufficiently sized drainage (outflow) channel. Contemporary optical and electronic components have reached minimal achievable dimensions, rendering camera and illuminating modules essentially fixed-scale. The realization of features (ii)-(iv) within a reduced insertion tube diameter thus delivers material advantages to patients, doctors, and manufacturers.

### SUMMARY OF THE DISCLOSURE

### Technical Problem

The present disclosure primarily addresses the technical challenge of minimizing the outer diameter of the insertion tube assembly while preserving: (i) an enlarged inner diameter of the instrument channel, (ii) a maximized irrigation (inflow) channel, and (iii) a maximized drainage (outflow) channel.

### Technical Solutions

According to a first aspect of the present disclosure, an insertion tube assembly for an endoscope is provided in some embodiments.

The insertion tube assembly for an endoscope may comprise:
a main-body segment, configured to connect to a distal end of a handle assembly of the endoscope, the main-body segment having a first channel disposed therein;
an open-port segment, connected to a distal end of the main-body segment; the open-port segment having a second channel and a third channel disposed therein, wherein the second channel and the third channel are isolated from each other, at least a portion of the second channel is arranged side-by-side with the third channel along a radial direction of the open-port segment; wherein an outer peripheral surface of the open-port segment is provided with an instrument outlet in communication with the second channel, and the third channel is in communication with the first channel;
a tip segment, connected to a distal end of the open-port segment; the tip segment having a fourth channel disposed therein, wherein a distal end face of the tip segment is provided with through-flow port in communication with the fourth channel, and a proximal end of the fourth channel is in communication with the third channel;
an imaging module, mounted on the tip segment; and
an instrument channel tube, disposed inside the first channel, wherein a distal end of the instrument channel tube is sealedly connected to a proximal end of the second channel, and a proximal end of the instrument channel tube is configured to connect to the handle assembly of the endoscope.

In some embodiments, a distal end of the second channel has a groove configuration forming the instrument outlet, a wall of the groove configuration forms an guiding slope surface configured to guide an instrument to extend out of the insertion tube assembly and to prevent the instrument from deflecting to both sides in a width direction of the groove configuration; the second channel and the third channel share a common partition layer, and the guiding slope surface is arranged on the portion.

In some embodiments, the fourth channel is radially offset toward one side of the tip segment, with its offset direction aligned with a direction of the instrument outlet; and the proximal end of the fourth channel opens toward the partition layer on its distal side.

In some embodiments, the groove configuration comprises a straight portion and an arcuate portion, the straight portion is disposed at a proximal side of the instrument outlet and extends along an axial direction of the open-port segment, and the arcuate portion is connected to a distal end of the straight portion.

In some embodiments, the outer peripheral surface of the tip segment is provided with a guiding groove, the guiding groove abuts distally against the groove configuration, and a wall of the guiding groove forms an extended guiding surface for bending guidance of an instrument.

In some embodiments, the guiding groove extends through the tip segment.

In some embodiments, a cross-section of the second channel on a side adjacent to the third channel is arcuate, thereby creating wedge-shaped spaces at circumferential ends of the third channel.

In some embodiments, the distal end face of the tip segment comprises an inclined portion, said inclined portion being disposed on at least one circumferential side of the distal end face, wherein at least a portion of the through-flow port is disposed on the inclined portion.

In some embodiments, the fourth channel is disposed on a radial side of the tip segment, the imaging module is disposed on another radial side of the tip segment; and a cross-section of the fourth channel is substantially an isosceles trapezoid, with a first base of said isosceles trapezoid proximal to the imaging module, and a second base distal to the imaging module.

In some embodiments, recesses are formed at two ends of the first base, said recesses being concave towards a side of the tip segment provided with the imaging module.

In some embodiments, the insertion tube assembly further comprises an illuminating module that is disposed on both sides of the imaging module along respective extension directions of the bases of the isosceles trapezoid.

In some embodiments, the main-body segment, the open-port segment and a port segment are three components connected in sequence, and the open-port segment is manufactured by injection molding, powder metallurgy, ceramic sintering, or 3D printing.

In some embodiments, the imaging module is connected to a connection line that extends through the third channel and the first channel, and a gap for liquid flow is provided between inner walls of the third and first channels and the connection line.

According to a second aspect of the present disclosure, an endoscope is provided in some embodiments.

The endoscope may comprise:
the insertion tube assembly according to any one of the embodiments mentioned above; and
a handle assembly, wherein a proximal end of the insertion tube assembly is coupled to the handle assembly, and the handle assembly is provided with an inflow port and a reflux port, with one of the inflow port and the reflux port being in communication with the proximal end of the instrument channel tube, and the other being in communication with space around the instrument channel tube within the first channel.

### Beneficial Effects

The beneficial effects of the present disclosure are as follows:

In the above-described endoscope, the radial side-by-side arrangement of the second and third channels within the insertion tube assembly, combined with the alignment of the second channel to the instrument outlet on its periphery and its connection to the instrument channel tube, enables instrument delivery while preventing radial dimension increase caused by the instrument channel tube. Furthermore, this arrangement permits dual functionality of the second channel as a fluid conduit supporting both inflow (for cavity irrigation) and return flow (for intracavitary reflux). Further, by disposing both the imaging module and the fourth channel on the distal end face of the insertion tube assembly (i.e., the distal end face of the tip segment) and integrating a through-flow channel that is positioned proximal to the imaging module and fluidly communicates with the fourth channel, the design enables selective implementation of return flow (intracavitary reflux) or inflow (cavity irrigation). Consequently, the integrated design achieves simultaneous diameter reduction and ensured fluid management functionality.

### BRIEF DESCRIPTION OF THE DRAWINGS

FIG. 1 is a perspective view of an endoscope in an embodiment of the present disclosure;

FIG. 2 is a perspective view of the insertion tube assembly in FIG. 1;

FIG. 3 is a longitudinal section view of FIG. 2;

FIG. 4 is an exploded view of the insertion tube assembly in FIG. 2;

FIG. 5 is a schematic diagram of the internal structure of the handle assembly in FIG. 1;

FIG. 6 is a longitudinal section view of FIG. 5;

FIG. 7 is a perspective view of an endoscope in another embodiment of the present disclosure;

FIG. 8 is a longitudinal section view of the insertion tube assembly in FIG. 7; and

FIG. 9 is an exploded view of the insertion tube assembly in FIG. 8.

### LIST OF REFERENCE NUMERALS

100 insertion tube assembly;
110 main-body segment;
120 open-port segment;
121 second channel;
122 guiding slope surface;
123 straight portion;
124 arcuate portion;
125 third channel;
126 instrument outlet;
130 tip segment;
131 fourth channel;
132 guiding groove;
133 inclined portion;
134 recess;
135 through-flow port;
136 fifth channel;
140 imaging module;
141 connection line;
150 instrument channel tube;
160 illuminating module;
200 handle assembly;
201 handle housing;
202 insertion tube mounting seat;
203 sealing adapter;
204 annular groove;
205 instrument access seal cap;
206 O-ring;
207 one-way duckbill valve;
208 one-way silicone pad;
209 inflow port;
210 reflux port;
300 fluid inflow/outflow assembly;
310 infusion pipe;
320 reflux pipe;
400 external cable.

### DETAILED DESCRIPTION

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusion is intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as ‘first,’ ‘second,’ or similar ordinal terms, serve solely to distinguish described objects and carry no inherent sequential or technical implications. Furthermore, the terms ‘connected’ and ‘coupled’ as used herein encompass both direct and indirect connection (coupling), unless explicitly stated otherwise.

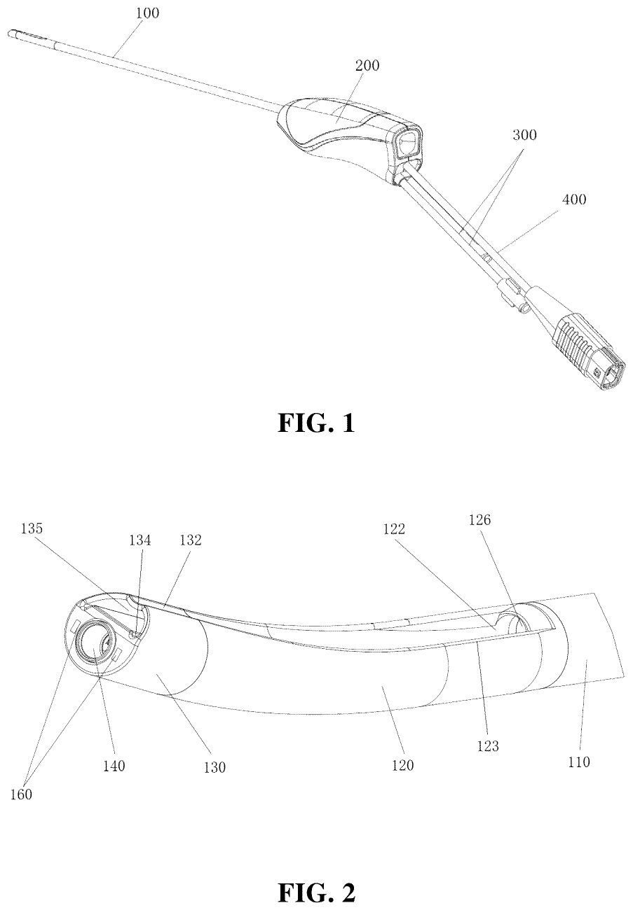

The endoscope according to some embodiments of the present disclosure may be a hysteroscope. Referring to FIG. 1, it comprises an insertion tube assembly 100, a handle assembly 200, a fluid inflow/outflow assembly 300, and an external cable 400. The insertion tube assembly 100 is adapted for insertion into a target position such as a body cavity, enabling visualization of the target position, and providing an instrument channel configured to guide a corresponding instrument. The handle assembly 200 may serve as a connection base of the insertion tube assembly 100, and is adapted to be gripped by an operator for manipulation. The fluid inflow/outflow assembly 300 is configured to regulate fluid circulation for cavity distension. The external cable 400 is configured to facilitate electrical transmission to support image output and power supply to the illuminating module 160. It should be noted that the endoscope disclosed herein may also be used in applications beyond hysteroscopy.

In an embodiment, the insertion tube assembly 100 for the endoscope includes a main-body segment 110, an open-port segment 120, and a tip segment 130. The main-body segment 110, typically constituting the longest portion of the insertion tube assembly 100, is operatively coupled to the distal end of the handle assembly 200 of the endoscope such that manipulation (e.g., movement or rotation) of the handle assembly 200 effectuates corresponding movement of the insertion tube assembly 100. In a specific embodiment, the main-body segment 110 may be configured as a cylindrical hollow tube body having an interior cavity that defines a first channel, said first channel being configured to receive and guide the instrument channel tube 150 therethrough and permit passage of the connection line 141. Specifically, the instrument channel tube 150 is disposed within the first channel, with its distal end sealedly connected to the proximal end of the second channel 121. The proximal end of the instrument channel tube 150 is configured to connect to the handle of the endoscope. Detailed description of the instrument channel tube 150 and the connection line 141 are set forth below.

The open-port segment 120 is connected to the distal end of the main-body segment 110. The open-port segment 120 has a second channel 121 and a third channel 125 that are disposed therein and are isolated from each other. At least a portion of the second channel 121 is arranged side-by-side with the third channel 125 along the radial direction of the open-port segment 120. The second channel 121 serves as an instrument channel and may also function as a fluid inflow or outflow channel. An instrument outlet 126, that is in communication with the second channel 121, is provided on the outer peripheral surface of the open-port segment 120. Instruments (e.g., biopsy forceps, scissors) inserted through the instrument channel tube 150 may extend out from the instrument outlet 126 to perform operations. The third channel 125 serves as a fluid outflow or inflow channel and as a channel for the connection line 141, allowing a cable connected to a corresponding module at the distal end of the insertion tube assembly 100 to be led out. The third channel 125 is connected to the first channel, allowing the third channel 125 to establish a connection to the handle assembly 200.

In an embodiment, to facilitate fabrication of the open-port segment 120, the main-body segment 110, the open-port segment 120, and a port segment are sequentially connected components. The open-port segment 120 is manufactured by injection molding, powder metallurgy, ceramic sintering, or 3D printing, the material being plastic, PEEK, metal, or the like. This provides simple processing, low cost, while guaranteeing dimensional tolerances, and sealing between the instrument channel and fluid channel.

To facilitate connection between the open-port segment 120 and the main-body segment 110, the outer peripheral surface of the proximal end of the open-port segment 120 comprises an outer stepped section with a diameter smaller than adjacent portions, configured to be press-fitted into the distal end of the main-body segment 110. To ensure unimpeded instrument passage, the inner periphery of the proximal end of the second channel 121 in the open-port segment 120 comprises an inner stepped section with a diameter larger than adjacent portions, enabling the inner wall of the instrument channel tube 150 to be flush with the inner wall of the proximal end of the second channel 121, eliminating any diameter discontinuity.

When the connection line 141 passes through the third channel 125, it occupies a portion of the space. To avoid affecting liquid infusion, the cross-section of a side of the second channel 121 adjacent to the third channel 125 is arcuate such that the circumferential ends of the third channel 125 forms a wedge-shaped space. This arcuate shape may be achieved by configuring the outer contour of the cross-section of the second channel 121 to be circular. In some embodiments, the connection line 141 may not be provided when a wireless imaging module 140 is used.

The tip segment 130 is connected to the distal end of the open-port segment 120. The tip segment 130 has a fourth channel 131 inside it. At the distal end face of the tip segment 130, a through-flow port 135 is provided for communicating with the fourth channel 131. The proximal end of the fourth channel 131 is in communication with the third channel 125. Providing the tip segment 130 allows installation of the imaging module 140 and facilitates infusion or reflux procedures via the through-flow port 135. Furthermore, positioning one of the infusion or reflux ports on the distal end face of the insertion tube assembly 100, and the other on a lateral side of the insertion tube assembly 100, promotes the establishment of a large liquid circulation loop (infusion/inflow and reflux/outflow). This enhances the efficiency of uterine distension, and prevents the situation where the liquid flows out from the infusion port and immediately flows out again when both the inflow and outflow ports are arranged on the distal end face. Additionally, configuring only one circulation port on the tip segment 130 achieves a larger liquid circulation area while controlling the diameter of the insertion tube assembly 100. This configuration is advantageous for improving liquid infusion efficiency.

It should be noted that the instrument outlet 126 provided on the outer peripheral surface of the insertion tube assembly 100 is configurable as an infusion port or a reflux port. Correspondingly, the through-flow port 135 provided at the distal end face of the insertion tube assembly 100 is configurable as the reflux port or the infusion port.

To facilitate insertion into natural orifices and prevent scraping of tissues or mucous membranes by sharp angles, the distal end face of the tip segment 130 includes an inclined portion 133 formed in at least one circumferential section thereof. At least a part of the through-flow port 135 is disposed on the inclined portion 133. In other embodiments, when the imaging module 140 is located centrally within the tip segment 130, the inclined portion 133 may be formed circumferentially to form a conical surface.

In an embodiment of the present disclosure, the fourth channel 131 is disposed along one radial section of the tip segment 130, while the imaging module 140 is disposed on another radial section of the tip segment 130. Accordingly, the cross-section of the fourth channel 131 has a non-standard geometry, substantially corresponding to an isosceles trapezoid. The first base of the isosceles trapezoid is proximate to the imaging module 140, and the second base is distal to the imaging module 140. This configuration optimally conforms to the cylindrical structure of the tip segment 130, thereby maximizing the cross-sectional area of the fourth channel 131. The term “substantially corresponding to an isosceles trapezoid” indicates that the overall contour of the cross-section approximately an isosceles trapezoid, while permitting: curved or non-linear legs, localized convex or concave deformations along the bases, and/or curved bases.

Further, in a specific embodiment, the first base is linear, with recesses 134 provided at both ends thereof. Said recesses 134 are concavely oriented toward the side of the tip segment 130 equipped with the imaging module 140. The recesses 134 may be configured as arc-shaped or rectangular profiles. In some embodiments, a fillet or chamfer may be formed between the bases and legs of the isosceles trapezoid. Additionally, in other embodiments, the cross-sectional shape of the fourth channel 131 may alternatively be of other configurations, such as but not limited to circular, triangular, semi-circular.

Where the illuminating module 160 is to be provided, the illuminating module 160 may be disposed along the extension direction of the bases on both sides of the imaging module 140, thereby enabling more effective utilization of the radial dimension of the tip segment 130 and enhanced control over the overall outer diameter of the insertion tube assembly 100.

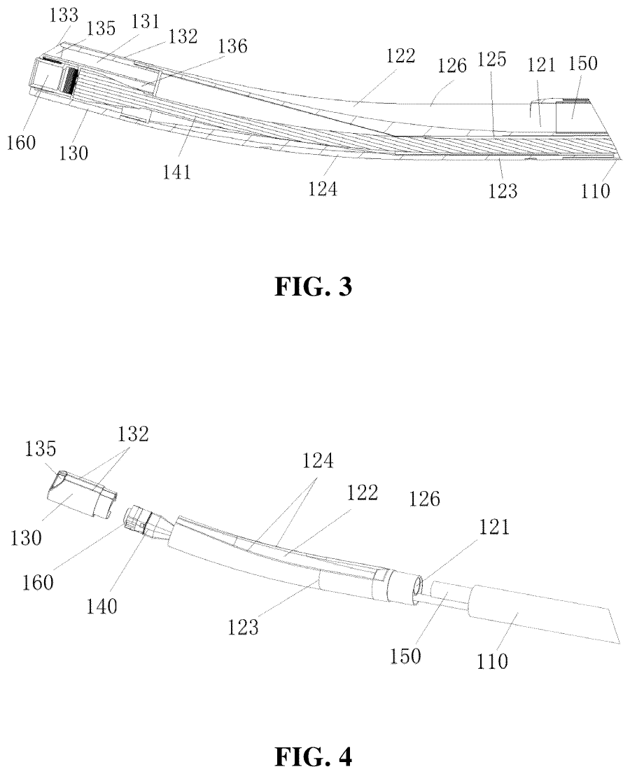

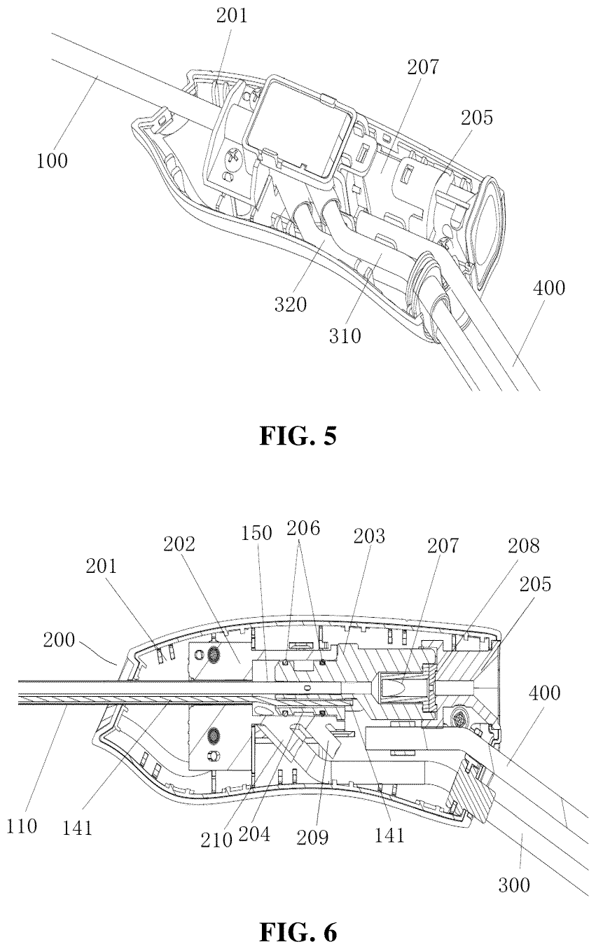

The tip segment 130 features mounting holes for installing the imaging module 140 and the illuminating module 160, with the remaining space configured as the fourth channel 131. In an embodiment, the imaging module 140 defines an imaging aperture on the distal end face of the tip segment 130 to capture images of the target position, while the illuminating module 160 provides illumination. The tip segment 130 may be made of transparent or semi-transparent materials. When constructed of transparent material, the imaging module 140 may be fully encapsulated within the tip segment 130. Referring to FIG. 3, in a specific embodiment, the tip segment 130 may be provided with a fifth channel 136 that is disposed radially adjacent to the fourth channel 131. The imaging module 140 is embedded in the fifth channel 136 and disposed at the distal end of the fifth channel 136. In other embodiments, the imaging module 140 has dimensions substantially matching the fifth channel 136, or the imaging module 140 is peripherally enveloped by the structural material of the tip segment 130 on all sides. Additionally, it should be noted that the imaging module 140 may be arranged coaxially with the tip segment 130 or offset radially to one side of the tip segment 130. In an exemplary embodiment, the imaging module 140 is offset to one side of the radial direction of the tip segment 130 and arranged in parallel with the fourth channel 131.

In an embodiment, the distal end of the second channel 121 comprises a groove configuration defining the instrument outlet 126. The wall of the groove configuration forms a guiding slope surface 122 configured to: guide an instrument radially outward relative to the insertion tube assembly 100, and confine lateral deviation of the instrument within the transverse width of the groove configuration. The groove configuration at the distal end of the second channel 121 functions as a retention mechanism, ensuring that the instrument advancing beyond the instrument channel maintains a prescribed trajectory into the endoscopic field of view, thereby preventing unintentional disengagement of the instrument from the groove configuration during rotation and avoiding internal tissue damage.

To facilitate manufacturing of the open-port segment 120, the second channel 121 and the third channel 125 share a common partition layer. The guiding slope surface 122 is disposed on the partition layer, while simultaneously allowing enlarged radial sizing for both the second channel 121 and the third channel 125.

With the provision of the partition layer, the fourth channel 131 is radially offset to one side of the tip segment 130, aligned with the orientation of the instrument outlet 126. The proximal end of the fourth channel 131 opens toward the distal side of the partition layer, thereby reducing flow resistance and facilitating smoother fluid discharge from or inflow into the proximal end of the fourth channel 131.

To enhance instrument guidance and mitigate disengagement risks, the groove configuration includes a straight portion 123 and an arcuate portion 124. The straight portion 123 is located on the proximal side of the instrument outlet 126 and extends along the axial direction of the open-port segment 120. The arcuate portion 124 is connected to the distal end of the straight portion 123. When the instrument is rotated, the straight portion 123 corresponding to the instrument outlet 126 accommodates greater structural compliance, thereby diminishing the force exertion on the distal end of the instrument and preventing the instrument from slipping out of the groove configuration.

Additionally, a guiding groove 132 is provided on the outer peripheral surface of the tip segment 130, and the guiding groove 132 is connected to the distal end of the groove configuration surface. The wall of the guiding groove 132 forms an extended guiding surface for guiding the instrument bending. The guiding groove 132 on the tip segment 130 allows longer-distance guidance of the instrument, improving the control of the instrument. Preferably, the guiding groove 132 extends through the tip segment 130, which not only improves the guiding effect but also positions the instrument closer to the axis of the insertion tube assembly 100, making it easier to rotate the instrument.

The handle assembly 200 comprises a handle housing 201, an insertion tube mounting seat 202, a sealing adapter 203, an instrument access seal cap 205, O-rings 206, a one-way duckbill valve 207, and a one-way silicone pad 208. The fluid inflow/outflow assembly 300 primarily includes an infusion pipe 310 and a reflux pipe 320. The proximal end of the main-body segment 110 of the insertion tube assembly 100 is fixedly connected and sealed to the inner hole at the front end of the insertion tube mounting seat 202. The sealing adapter 203 is inserted into the proximal end of the insertion tube mounting seat 202 and sealed with the insertion tube mounting seat 202 by means of two O-rings 206. A ring groove 204 is provided on the sealing adapter 203 between the two O-rings 206 to form a reflux cavity. The bottom wall/surface of the ring groove 204 is provided with a radial through hole placing the reflux cavity in fluid communication with the reflux port 210. The reflux pipe 320 communicates with the reflux cavity via the reflux port 210. Radial through holes may also be provided on the outer periphery of the instrument channel tube 150, placing the instrument channel tube 150 in fluid communication with the reflux cavity. The distal end of the sealing adapter 203 and the insertion tube assembly 100 together define an infusion cavity. The infusion water pipe 310 communicates with the infusion cavity through the inflow port 209, thereby enabling infusion liquid circulation. The rear end of the sealing adapter 203 is provided with an inner hole for mounting the one-way duckbill valve 207 and the one-way silicone pad 208. The instrument access seal cap 205 is then mounted to the rear end of the sealing adapter 203 and clamps the one-way duckbill valve 207 and the one-way silicone pad 208, causing them to tightly engage with a step of the sealing adapter 203. The one-way duckbill valve 207 is a one-way valve structure. When no instrument is inserted into the endoscope, this valve prevents liquid in the instrument channel tube 150 from flowing back to the instrument access seal cap 205. When an instrument is inserted into the endoscope, the one-way duckbill valve 207 is opened, and the outer diameter of the handle of the instrument tightly engages with the inner hole of the one-way silicone pad 208, preventing liquid in the instrument channel tube 150 from flowing back to the instrument access seal cap 205. Thus, whether or not an instrument is inserted into the endoscope, liquid in the instrument channel tube 150 is prevented from flowing back to the instrument access seal cap 205. Liquid can only be discharged from the human body and the endoscope via the reflux cavity and the reflux pipe 320. The other end of the connection cable 141 is provided with a plug capable of being quickly connected to an endoscope image processor, thereby enabling signal transmission and control between the imaging module 140, the illuminating module 160 and the endoscope image processor.

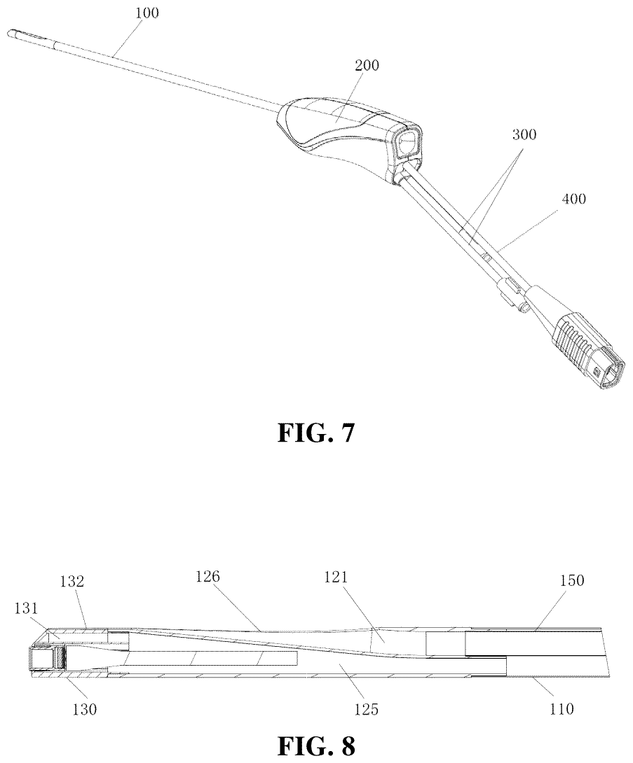

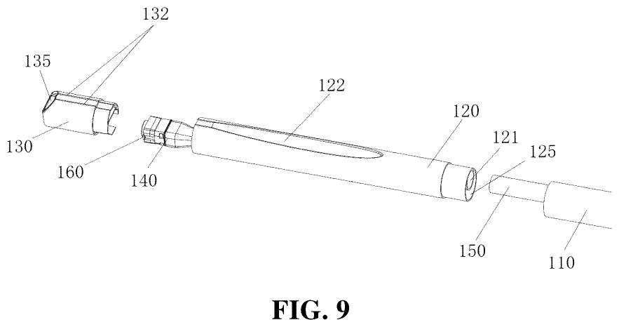

In the above embodiments, the open-port segment 120 of the insertion tube assembly 100 is a curved structure. In other embodiments, referring to FIGS. 7 to 9, the open-port segment 120 of the insertion tube assembly 100 may also be a linear structure.

During use, the second channel 121 may function as an instrument channel, so that the instrument channel does not pass through the interior of the tip segment 130. Under the premise of ensuring the same inner diameter of the instrument channel, the outer diameter of the insertion tube assembly 100 can be significantly reduced. Concurrently, the second channel 121 may also function as a channel for inflow or reflux, while the fourth channel 131, the third channel 125, and the first channel can cooperate to serve as the channel for reflux or inflow (as designated). These flow paths have large cross-sectional areas, resulting in higher infusion efficiency. Additionally, the distance between the water inflow and reflux port can be increased, which better establishes the irrigation flow loop, reduces patient discomfort, and facilitates doctors' operations.

Embodiments of the insertion tube assembly of the endoscope in the present disclosure:

The structure of the insertion tube assembly of the endoscope is the same as that of the insertion tube assembly 100 described in any embodiment of the above-mentioned endoscope, and will not be described in detail here.

The specific embodiments described herein are presented only to illustrate the principle of the present disclosure and shall not be construed as limiting the scope of the present disclosure. Those skilled in the art may effect variations, modifications, or equivalent substitutions based on the inventive principle disclosed herein, without departing from the spirit and scope of the disclosure.

1. An insertion tube assembly for an endoscope, comprising:
a main-body segment, configured to connect to a distal end of a handle assembly of the endoscope, the main-body segment having a first channel disposed therein;
an open-port segment, connected to a distal end of the main-body segment; the open-port segment having a second channel and a third channel disposed therein, wherein the second channel and the third channel are isolated from each other, at least a portion of the second channel is arranged side-by-side with the third channel along a radial direction of the open-port segment; wherein an outer peripheral surface of the open-port segment is provided with an instrument outlet in communication with the second channel, and the third channel is in communication with the first channel;
a tip segment, connected to a distal end of the open-port segment; the tip segment having a fourth channel disposed therein, wherein a distal end face of the tip segment is provided with through-flow port in communication with the fourth channel, and a proximal end of the fourth channel is in communication with the third channel;
an imaging module, mounted on the tip segment; and
an instrument channel tube, disposed inside the first channel, wherein a distal end of the instrument channel tube is sealedly connected to a proximal end of the second channel, and a proximal end of the instrument channel tube is configured to connect to the handle assembly of the endoscope.

2. The insertion tube assembly for an endoscope according to claim 1, wherein
a distal end of the second channel has a groove configuration forming the instrument outlet,
a wall of the groove configuration forms a guiding slope surface configured to guide an instrument to extend out of the insertion tube assembly and to prevent the instrument from deflecting to both sides in a width direction of the groove configuration;
the second channel and the third channel share a common partition layer, and
the guiding slope surface is arranged on the portion.

3. The insertion tube assembly for an endoscope according to claim 2, wherein
the fourth channel is radially offset toward one side of the tip segment, with its offset direction aligned with a direction of the instrument outlet; and
the proximal end of the fourth channel opens toward the partition layer on its distal side.

4. The insertion tube assembly for an endoscope according to claim 2, wherein
the groove configuration comprises a straight portion and a arcuate portion,
the straight portion is disposed at a proximal side of the instrument outlet and extends along an axial direction of the open-port segment, and
the arcuate portion is connected to a distal end of the straight portion.

5. The insertion tube assembly for an endoscope according to claim 2, wherein
the outer peripheral surface of the tip segment is provided with a guiding groove,
the guiding groove abuts distally against the groove configuration, and
a wall of the guiding groove forms an extended guiding surface for bending guidance of an instrument.

6. The insertion tube assembly for an endoscope according to claim 5, wherein
the guiding groove extends through the tip segment.

7. The insertion tube assembly for an endoscope according to claim 1, wherein
a cross-section of the second channel on a side adjacent to the third channel is arcuate, thereby creating wedge-shaped spaces at circumferential ends of the third channel.

8. The insertion tube assembly for an endoscope according to claim 1, wherein
the distal end face of the tip segment comprises an inclined portion,
said inclined portion being disposed on at least one circumferential side of the distal end face,
wherein at least a portion of the through-flow port is disposed on the inclined portion.

9. The insertion tube assembly for an endoscope according to claim 1, wherein
the fourth channel is disposed on a radial side of the tip segment,
the imaging module is disposed on another radial side of the tip segment; and
a cross-section of the fourth channel is substantially an isosceles trapezoid, with a first base of said isosceles trapezoid proximal to the imaging module, and a second base distal to the imaging module.

10. The insertion tube assembly for an endoscope according to claim 9, wherein
recesses are formed at two ends of the first base,
said recesses being concave towards a side of the tip segment provided with the imaging module.

11. The insertion tube assembly for an endoscope according to claim 9, further comprising:
an illuminating module, disposed on both sides of the imaging module along respective extension directions of the bases of the isosceles trapezoid.

12. The insertion tube assembly for an endoscope according to claim 1, wherein
the main-body segment, the open-port segment and a port segment are three components connected in sequence, and
the open-port segment is manufactured by injection molding, powder metallurgy, ceramic sintering, or 3D printing.

13. The insertion tube assembly for an endoscope according to claim 1, wherein
the imaging module is connected to a connection line that extends through the third channel and the first channel, and
a gap for liquid flow is provided between inner walls of the third and first channels and the connection line.

14. An endoscope, comprising:
the insertion tube assembly according to claim 1; and
a handle assembly, wherein a proximal end of the insertion tube assembly is coupled to the handle assembly, and the handle assembly is provided with an inflow port and a reflux port, with one of the inflow port and the reflux port being in communication with the proximal end of the instrument channel tube, and the other being in communication with space around the instrument channel tube within the first channel.
